(19) United States
(12) Patent Application Publication
(10) Pub. No.: US 2021/0275804 A1
(43) Pub. Date: Sep. 9, 2021

(54) Wire Lead for Neural Electrical Stimulation

(71) Applicant: Amitabh Goel, Lakewood Ranch, FL (US)

(72) Inventor: Amitabh Goel, Lakewood Ranch, FL (US)

(21) Appl. No.: 17/326,735

(22) Filed: May 21, 2021

(51) Int. Cl. A61N 1/05; A61N 1/375

(52) U.S. Cl. CPC A61N 1/0558; A61N 1/375

(57) ABSTRACT

A wire lead for neural electrical stimulation, the wire lead having a plurality of interior electrical conductors; the wire lead further having a plastic sheath having an axial end, an oppositely axial end, and a radially outer end, the plurality of electrical conductors extending through the plastic sheath from the oppositely axial end to the axial end; the wire lead further having a multiplicity of axially facing lands fixedly attached to or formed wholly with the radially outer end of the plastic sheath; the wire lead further having a multiplicity of oppositely axially facing lands, wherein each land among the multiplicity of oppositely axially facing lands is positioned between an adjacent pair of the axially facing lands.

### FIELD OF THE INVENTION

This invention relates to apparatus and assemblies which are adapted to suppress or eliminate bodily pain by facilitating applications of electrical voltages to and inducement of electrical currents within a nerve, commonly the spinal cord would, in the absence of induced electrical currents, conduct such pain. More particularly, this invention relates to configurations of percutaneously implanted electrically conductive wire leads which transmit such voltage and current.

### BACKGROUND OF THE INVENTION

Commonly known assemblies and mechanisms for electric nerve stimulation effected pain suppression include a compact percutaneously implanted battery pack or generator which houses electronic circuitry for generation of low voltage electric pulses. The generator component of the assembly communicates with an attached electrically conductive wire lead. In many instances, the generator is surgically implanted percutaneously at the lower back of a patient who experiences uncontrolled pain.

The electrically conductive wire lead component of the assembly extends distally or axially from the generator a short distance immediately subcutaneously. Such wire's axial extension then typically traverses underlying bodily tissues including subcutaneous adipose, back muscles, and spinal ligaments, and finally enters, for example, the patient's spinal canal. Such electrically conductive wire lead is commonly further extended axially along the spinal canal and within the epidural space in relation to the spinal cord. The initial passage and extension of the wire lead is typically accomplished through the use of a carefully targeted and extended hollow bored Tuohy needle which initially pierces such tissue layers and guides the wire lead therethrough.

Electrically conductive contact electrodes are commonly mounted and exposed at the extreme distal or axial end of the electrically conductive wire lead. The above described wire lead extension along the epidural space terminates when such electrodes directly overlie and align with a selected electrical stimulation site along the patient's spinal cord. Similar wire lead extensions and electrode placements commonly occur in relation to a pain patient's peripheral spinal nerves.

Flexible twisting, bending, stretching and retracting motions of the tissues through which the electrically conductive wire lead extends are known to produce undesirable axial and oppositely slipping movements of the wire lead, such slippage potentially disrupting the targeted placement of nerve stimulating electrodes. Such motions often extend or retract the wire lead away from its originally selected and desired placement, undesirably dragging the electrodes at the distal end of the wire lead away from their originally selected electrical stimulation site. Such wire slip induced disruption of the selected electrode placement may render the apparatus unable to ameliorate the patient's pain.

The instant inventive wire lead for neural electrical stimulation solves or ameliorates the above described wire slip related problem and complications experienced with use of conventional electrical stimulator wire leads by specially configuring the wire lead's sheathing to present multiplicities of axially and oppositely axially facing lands which operatively engage and anchor against tissues surrounding the wire lead.

### BRIEF SUMMARY OF THE INVENTION

The instant inventive wire lead is intended for use in pain suppressing neural electrical stimulation at nerve sites, including a pain patient's spinal nerve or one of such patient's peripheral spinal nerves. A first structural component of the instant inventive wire lead comprises a plurality of wire configured electrical conductors. Axial or distal ends of such conductors are preferably attached in electrical communication with a series of small tissue contacting electrodes. The proximal or oppositely axial ends of such electrical conductors electrically attached to and communicate with a battery powered electrical pulse generating pack or unit. Such battery powered pulse generating pack is commonly surgically implanted upon a pain patient, the generator commonly being sited percutaneously at the small of the patient's back.

A further structural component of the instant inventive wire lead comprises a flexible plastic sheath which surrounds and protects the electrical conductors, such sheath extending along the axial length of the conductors from the generator to the electrodes. In a preferred embodiment, the radially inner matrix of the plastic sheath internally supports and suspends the electrical conductors while separating and insulating the electrical conductors with respect to each other. The insulating function of the sheath assures that an electric pulse applies to the proximal end of one of the conductors exclusively grounds at the electrode attachment to that conductor's opposite end.

Further structural components of the instant inventive lead comprise a multiplicity of axially facing lands, each of which is fixedly attached to or formed wholly with the radially outer end of the flexible plastic sheath. Such multiplicity of lands are preferably sited along the length of the flexible sheet to correspond with a portion of the wire lead which angularly approaches the nerve which is to be electrically stimulated.

Further structural components of the instant inventive wire lead comprise a multiplicity of oppositely axially facing lands, each of which is preferably being positioned between an adjacent pair of the axially facing lands.

In a preferred embodiment of the instant inventive wire lead, the multiplicities of axially facing and oppositely axially facing lands are positioned along the plastic sheath so that upon an extension of the plastic sheath, including its conductors and distal end electrodes, to a targeted electrical stimulation site along the spinal cored or peripheral nerve, the axially and oppositely axially facing lands reside at a site where healing formations of collagen tissue scarring may naturally occur. Prior to the commencement of healing of tissue disruptions which occur upon the initial implant of the wire lead, the multiplicities of axial facing and oppositely facing lands beneficially frictionally engage surrounding tissues to provide traction against unwanted axial or oppositely axial slippage of the wire lead. Subsequently, healing formations of collagen fiber scar tissue scarring advantageously bias against and mechanically engage the multiplicities of axial facing and oppositely axial facing lands, effectively permanently anchoring the wire lead and assuring that the lead will not disrupt the targeted electrode placement.

In preferred embodiments of the instant inventive wire lead, the axially facing and oppositely axially facing lands extend either circumferentially about or helically along the sheath and its electrical conductors.

Accordingly, objects of the instant invention include the provision of a wire lead for neural electrical stimulation which incorporates structures, as described above, and which arranges those structures in relation to each other in the manners described above, for the performance of beneficial functions as described above.

Other and further objects, benefits, and advantages of the instant invention will become known to those skilled in the art upon review of the Detailed Description which follows, and upon review of the appended drawings.

### BRIEF DESCRIPTION OF THE DRAWINGS

FIG. 1 presents a sectional view of the spine of a patient who experiences uncontrolled pain, the view showing an exemplary extension of and placement of the instant inventive wire lead.

FIG. 2 is a magnified view of an exemplary segment or length of the inventive wire lead, the view showing in dashed lines internal electrical conductive wires.

FIG. 3 is a magnified view of a portion of the structure of FIG. 1, as indicated in FIG. 1.

FIG. 4 is a magnified view of a portion of the structure of FIG. 3, as indicated in FIG. 3.

FIG. 5 redepicts the structure of FIG. 2, the view of FIG. 5 alternatively showing helically extending land and valley components.

### DETAILED DESCRIPTION OF A SUITABLE EMBODIMENT

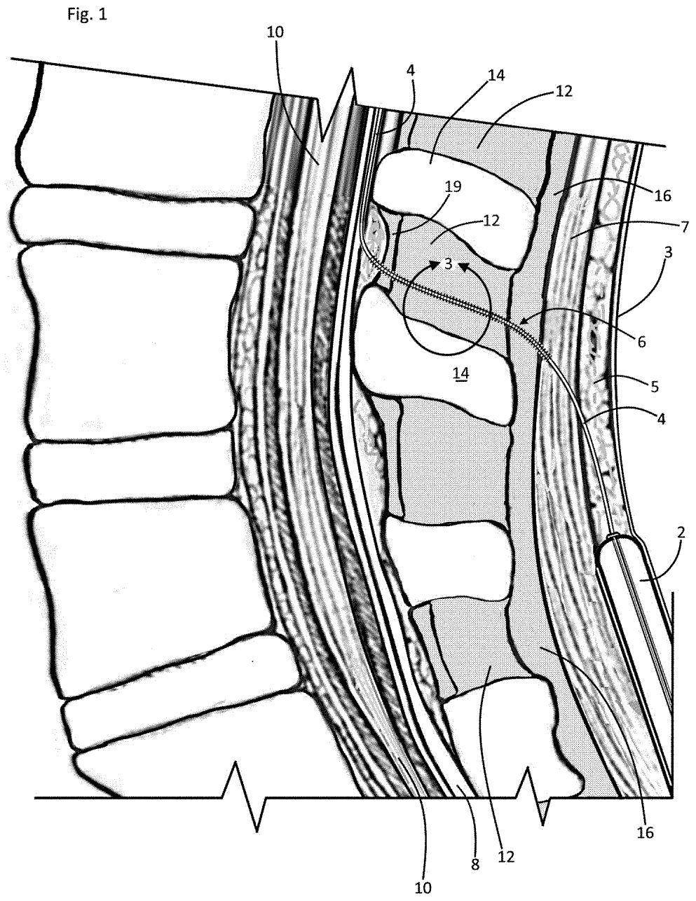

Referring now to the drawings, and in particular to Drawing FIG. 1, a suitable embodiment of the instant inventive wire lead for neural electrical stimulation is referred to generally by Reference Arrow 6. Such wire lead 6 suitably constitutes a segment or length of a conventional electrical stimulator wire lead 4 which is served by and extends axially or distally from a percutaneously implanted electric pulse generator pack 2. As shown in the exemplary implantation of Drawing FIG. 1, the electrical pulse generator pack 2 immediately underlies a pain patient's skin 3, the pack 2 commonly being positioned at the small of the patient's back. The pack 2 is held within an adipose tissue layer 5, immediately dorsally overlying the patient's back muscles 7. The wire lead 4 emerges at an axial end of the generator pack 2, then angularly traversing the adipose layer 5. The land 4 then traverses back muscles 7, and then traverses, for example, the patient's supraspinous ligament 16. Further progression toward the patient's spinal cord 10 may extend the lead 4 along a traversing path through one of the patient's interspinous ligaments 12, then traversing a caudally underlying spinous ligamentum flavum 19. Finally, the extension of the lead 4 enters the patient's epidural space 8. The initial installation of the wire lead 4 through such tissue layers 5, 7, 16, 17, and 19 is commonly effects and guided by a Tuohy needle which lances through such tissue layers.

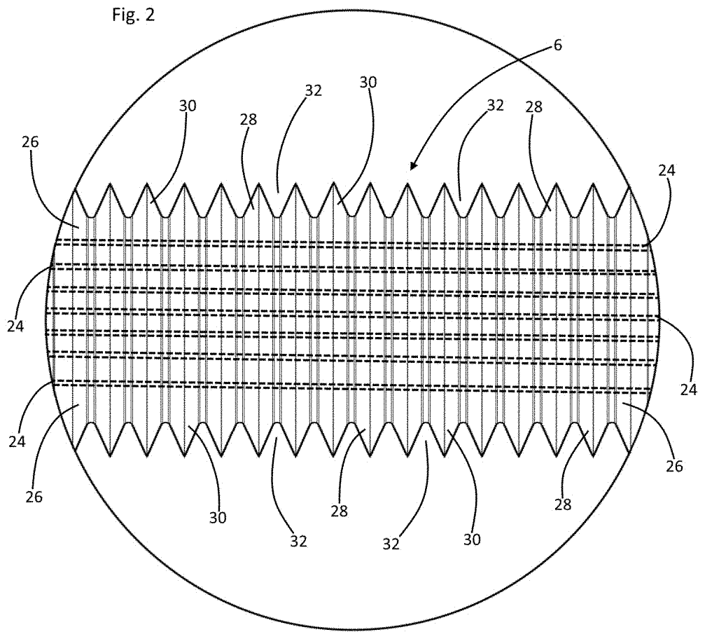

Threading of the wire lead 4 through such needle preferably dispenses the wire lead 4 into the epidural space 8 until an extension is reached at which the electrodes mounted at the extreme axial or distal end of the lead reside at and immediately dorsally overlie a selected spinal cord electrical stimulation site. Thereafter, such Tuohy needle may be proximally withdrawn from the wire lead 4. Thereafter, referring further simultaneously to Drawing FIGS. 1 and 2, electrically conductive wires 24 extending form the proximal end of the wire lead 4 are attached in electrical communication with electric outlet contacts of the electrical pulse generating pack or unit 2. Following testing and programming to produce optimum pain suppressing electrical pulse lengths, strengths, and intervals, the pulse generator pack 2 may be installed percutaneously beneath skin 3 as indicated in FIG. 1.

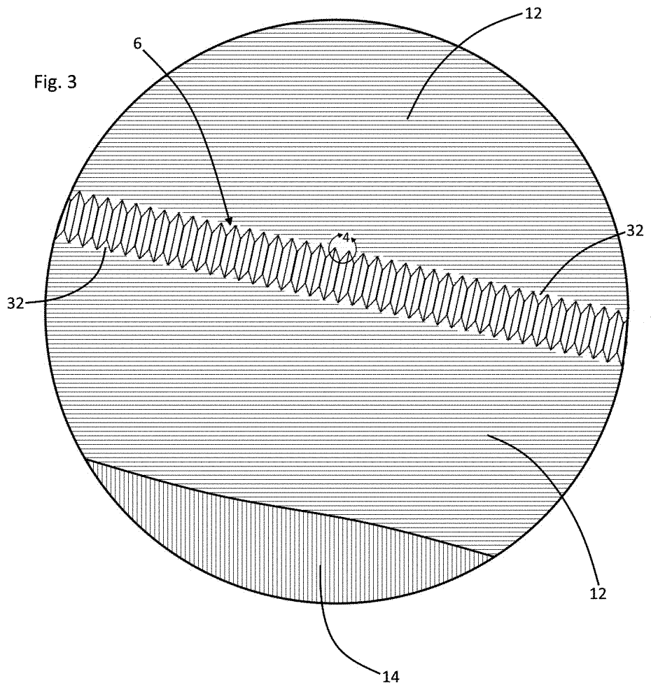

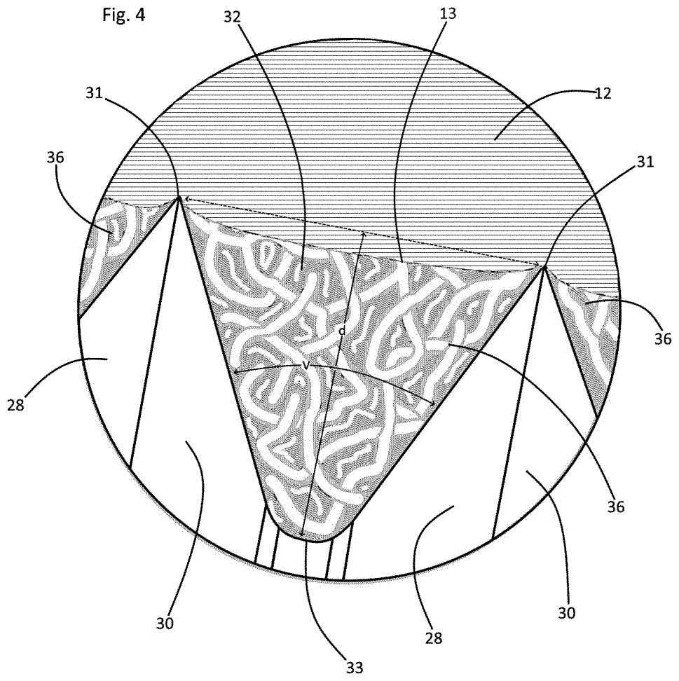

Referring to FIG. 2, the instant invention's specialized wire lead segment 6 suitably includes a flexible plastic sheath 26 which annularly surrounds and extends axially and oppositely axially along wire electrical conductors 24, such conductors being suspended and respectively electrically insulated at the inner plastic matrix of the sheath 26. Referring further simultaneously to FIGS. 3 and 4, a multiplicity of axially facing lands 28 is fixedly attached to or is formed wholly with the plastic sheath 26, such lands 28 performing functions which are further described in detail below.

Further structural components of the instant inventive wire lead comprise a multiplicity of oppositely axially facing lands or proximal facing lands 30, each land 30 preferably residing between an adjacent pair of the axially facing lands 28. The depicted arrangement of axially facing and oppositely facing lands 28 and 30 beneficially forms a multiplicity of knife edge shaped ridges 28,30, each such ridge suitably extending circumferentially about the conductors 24 at the interior of the sheath 26. The circumferentially extending ridges 28,30 beneficially form a multiplicity of circumferentially extending valleys 32 each of which is axially bounded by one of the oppositely axially facing lands 30 and is oppositely axially bounded by one of the axially facing lands 28.

Referring in particular to FIG. 4, in a suitable embodiment, the lands 28 and 30 extend radially from the axial center of the wire lead distances “d” between ridge apexes 31 and the floors 33 of the valleys 32. In the preferred embodiment, the depths “d” of the valleys 32 is between 0.1 millimeters and 0.5 millimeters. Also in the suitable embodiment, the angle “v” between the axially paired lands 28 and 30 is between 30° and 60°.

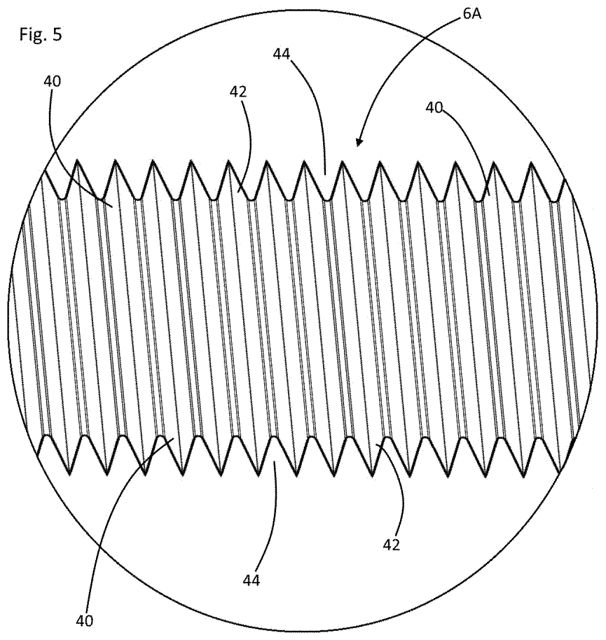

Referring simultaneously to FIGS. 2 and 5, an alternatively configured wire lead segment 6A is configured similarly with the wire lead segment 6 of the FIG. 2 embodiment with the exception that a single helically extending and axially facing land 40 is provided in place of FIG. 2's multiple circumferential axially facing lands 28. FIG. 5's helical oppositely axially extending land 42 in conjunction with land 40 forms a single helically extending “V” valley 44 which functions similarly with the multiple valleys 32 of the Drawing FIG. 2 embodiment.

In operation of the instant inventive electrical stimulation wire lead 6 or 6A, as the case may be, and referring in particular to Drawing FIG. 4, a Tuohy needle (not depicted within views) may initially pass through spinal ligament tissue such as an interspinous ligament 12. During its insertion, such Tuohy needle cuts a cylindrical channel having a cylindrical wall designated by Reference Numeral 13. Upon the initial implantation of the wire lead segment 6 within the channel formed by the Tuohy needle, apexes or ridge edges 31 which are formed by the axial and oppositely axial lands 28 and 30 initially bear against and frictionally engage the ligament tissue wall 13. Such frictional engagements of the apexes 31 against the ligament wall 13 advantageously anchors the wire lead in place.

Following such frictional apex 31 and wall 13 engagements, healing and collagen scar tissue 36 formation naturally occurs within the valleys 32 which are formed between the lands 28 and 30. Such collagen fiber scar formation advantageously further anchors the wire lead against axial slippage.

While the principles of the invention have been made clear in the above illustrative embodiment, those skilled in the art may make modifications to the structure, arrangement, portions and components of the invention without departing from those principles. Accordingly, it is intended that the description and drawings be interpreted as illustrative and not in the limiting sense, and that the invention be given a scope commensurate with the appended claims.

The invention hereby claimed is:

1. A wire lead for neural electrical stimulation, the wire lead comprising:
(a) a plurality of electrical conductors;
(b) a plastic sheath having an axial end, an oppositely axial end, and a radially outer end, the plurality of electrical conductors extending through the plastic sheath from its oppositely axial end to its axial end;
(c) a multiplicity of axially facing lands, each land among the multiplicity of axially facing lands being fixedly attached to or formed wholly with the radially outer end of the plastic sheath; and
(d) a multiplicity of oppositely axially facing lands, each land among the multiplicity of oppositely axially facing lands being positioned between an adjacent pair of the axially facing lands.

2. The wire lead for neural electrical stimulation of claim 1 wherein each land among the multiplicities of axially facing and oppositely axially facing lands extends circumferentially about the plurality of electrical conductors.

3. The wire lead for neural electrical stimulation of claim 2 further comprising a multiplicity of valleys, each valley extending circumferentially about the plurality of electrical conductors, said each valley being oppositely axially bounded by one of the axially facing lands, said each valley being axially bounded by one of the oppositely axially facing lands.

4. The wire lead for neural electrical stimulation of claim 3 wherein said each valley has a depth between 0.1 millimeters and 0.5 millimeters.

5. The wire lead for neural electrical stimulation of claim 4 wherein said each valley has a “V” angle between 30° and 60°.

6. A wire lead for neural electrical stimulation, said wire lead comprising:
(a) a plurality of electrical conductors;
(b) a plastic sheath having an axial end, an oppositely axial end, and a radially outer end, the plurality of electrical conductors extending through the plastic sheath from its oppositely axial end to its axial end;
(c) an axially facing land fixedly attached to or formed wholly with the radially outer end of the plastic sheath, said land extending helically about the plastic sheath; and
(d) an oppositely axially facing land fixedly attached to or formed wholly with the radially outer end of the plastic sheath, said land extending helically about the plastic sheath.

7. The wire lead for neural electrical stimulation of claim 6 further comprising a helically extending valley, said valley being oppositely axially bounded by the axially facing land, said land being axially bounded by the oppositely axially facing land.

8. The wire lead for neural electrical stimulation of claim 7 wherein the helically extending valley has a depth between 0.1 millimeters and 0.5 millimeters.

9. The wire lead for neural electrical stimulation of claim 8 wherein the helically extending valley has a “V” angle between 30° and 60°.
